(19) United States
(12) Patent Application Publication
(10) Pub. No.: US 2022/0395490 A1
(43) Pub. Date: Dec. 15, 2022

(54) TREATMENT OF ARTHRITIS

(71) Applicant: Inflazome Limited, Dublin 2 (IE)

(72) Inventors: Matthew COOPER, Cambridge (GB); Luke Anthony John O'NEILL, Dublin 2 (IE)

(21) Appl. No.: 17/775,243

(22) PCT Filed: Nov. 6, 2020
(86) PCT No.: PCT/EP2020/081291

§ 371 Date: May 6, 2022

(30) Foreign Application Priority Data
Nov. 7, 2019 (GB) 1916237.9

(51) Int. Cl. A61K 31/415; A61P 19/02; A61P 19/06

(52) U.S. Cl. CPC A61K 31/415; A61P 19/02; A61P 19/06; C07B 2200/13; A61K 9/0053

(57) ABSTRACT

The present invention relates to a compound of formula (I) for use in the treatment or prevention of arthritis.

The present invention relates to a compound of formula (I):

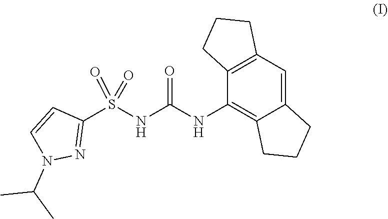 [SMILES: CC(C)n1ccc(S(=O)(=O)NC(=O)Nc2c3c(cc4c2CCC4)CCC3)n1]

for use in the treatment or prevention of arthritis.

Arthritis includes gout, pseudogout, rheumatoid arthritis and osteoarthritis.

NLRP3 has been implicated in the pathogenesis of gout and pseudogout (Wen et al., Nature Immunology, 13: 352-357, 2012; Duewell et al., Nature, 464: 1357-1361, 2010; Strowig et al., Nature, 481: 278-286, 2012; and Klück et al., The Lancet Rheumatology, 2(5): E270-E280, 2020). Gout is a relatively common disease, with a prevalence of 1-2% in Europe, and 3-4% in the USA. Current drug therapies focus on anti-inflammatory/analgesic treatment and the lowering of the serum urate concentration. In the chronic treatment phase, there is a sub-population of patients that do not respond properly to maximum treatment. This can be due to hypersensitivity to the drug or adverse events. These patients, called ‘refractory gout patients’, have a significant unmet need. Pseudogout is thought to be mediated by calcium-pyrophosphate crystals which activate NLRP3.

Osteoarthritis (OA) is the most common form of arthritis in the world affecting approximately 3.5% of the population. Osteoarthritis is caused when inflammatory proteins and proteases cause joint destruction. NLRP3 activation has been shown to drive the inflammatory component and its inhibition may arrest disease progression (Jin et al., PNAS, 108(36): 14867-14872, 2011; Guo et al., Clin Exp Immunol, 194(2): 231-243, 2018; Braddock et al., Nat Rev Drug Disc, 3: 1-10, 2004; McAllister et al., Osteoarthritis and Cartilage, 26(5): 612-619, 2018; and Ridger et al., New England J Medicine, 377: 1119-1131, 2017).

Rheumatoid arthritis (RA) is also relatively common, affecting approximately 1% of the population, and rheumatoid arthritis has also been shown to involve NLRP3 (Masters, Clin Immunol, 147(3): 223-228, 2013; Braddock et al., Nat Rev Drug Disc, 3: 1-10, 2004; Inoue et al., Immunology, 139: 11-18, 2013; Scott et al., Clin Exp Rheumatol, 34(1): 88-93, 2016; Guo et al., Clin Exp Immunol, 194(2): 231-243, 2018; and Dong et al., Cellular & Molecular Immunology, 17: 261-271, 2020).

This invention is based on the discovery that the compound of formula (I) is particularly effective in the treatment of arthritis, most especially via the oral route.

In a first aspect of the present invention, there is provided a compound of formula (I):

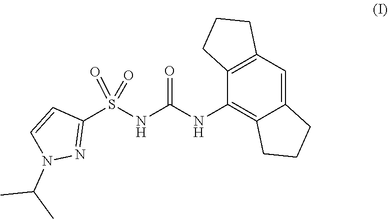 [SMILES: CC(C)n1ccc(S(=O)(=O)NC(=O)Nc2c3c(cc4c2CCC4)CCC3)n1]

or a pharmaceutically acceptable salt thereof, for use in the treatment or prevention of arthritis.

In one embodiment, the arthritis is gout such as refractory gout. In another embodiment, the arthritis is pseudogout. In another embodiment, the arthritis is rheumatoid arthritis. In another embodiment, the arthritis is osteoarthritis.

In one embodiment, the treatment or prevention comprises the treatment or prevention of inflammation. Typically, the treatment or prevention of inflammation is achieved via NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

In one embodiment, the treatment or prevention comprises the oral administration of the compound or the salt thereof.

In one embodiment, the compound or salt is a sodium salt, such as a monosodium salt.

In one embodiment, the compound or salt is a monohydrate. In one embodiment, the compound or salt is crystalline. In one embodiment, the compound or salt is a crystalline monosodium monohydrate salt. In one embodiment, the crystalline monosodium monohydrate salt has an XRPD spectrum comprising peaks at: 4.3° 2θ, 8.7° 2θ, and 20.6° 2θ, all ±0.2° 2θ. In one embodiment, the crystalline monosodium monohydrate salt has an XRPD spectrum in which the 10 most intense peaks include 5 or more peaks which have a 2θ value selected from: 4.3° 2θ, 6.2° 2θ, 6.7° 2θ, 7.3° 2θ, 8.7° 2θ, 9.0° 2θ, 12.1° 2θ, 15.8° 2θ, 16.5° 2θ, 18.0° 2θ, 18.1° 2θ, 20.6° 2θ, 21.6° 2θ, and 24.5° 2θ, all ±0.2° 2θ. The XRPD spectrum may be obtained as described in WO 2019/206871, which is incorporated in its entirety herein by reference.

In one embodiment, the crystalline monosodium monohydrate salt is as described in WO 2019/206871, which is incorporated in its entirety herein by reference. In one embodiment, the crystalline monosodium monohydrate salt has the polymorphic form described in WO 2019/206871, which is incorporated in its entirety herein by reference. In one embodiment, the crystalline monosodium monohydrate salt is prepared according to the method described in WO 2019/206871, which is incorporated in its entirety herein by reference.

In a second aspect of the present invention, there is provided a pharmaceutical composition comprising a pharmaceutically acceptable excipient and a compound or salt of the first aspect of the present invention. In one embodiment, the pharmaceutical composition is suitable for oral administration.

In a third aspect of the present invention, there is provided a method for the treatment or prevention of arthritis in a patient in need thereof, wherein the method comprises administering to the patient in need thereof a therapeutically or prophylactically effective amount of a compound of formula (I):

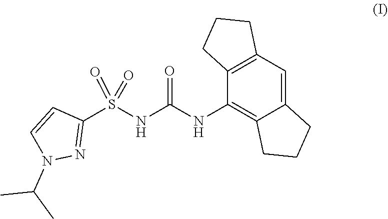 [SMILES: CC(C)n1ccc(S(=O)(=O)NC(=O)Nc2c3c(cc4c2CCC4)CCC3)n1]

or a pharmaceutically acceptable salt thereof.

In one embodiment, the arthritis is gout such as refractory gout. In another embodiment, the arthritis is pseudogout. In another embodiment, the arthritis is rheumatoid arthritis. In another embodiment, the arthritis is osteoarthritis.

In one embodiment, the treatment or prevention comprises the treatment or prevention of inflammation. Typically, the treatment or prevention of inflammation is achieved via NLRP3 inhibition.

In one embodiment, the treatment or prevention comprises the oral administration of the compound or the salt thereof.

In one embodiment, the compound or salt is a sodium salt, such as a monosodium salt. In one embodiment, the compound or salt is a monohydrate. In one embodiment, the is compound or salt is crystalline. In one embodiment, the compound or salt is a crystalline monosodium monohydrate salt. In one embodiment, the crystalline monosodium monohydrate salt has an XRPD spectrum comprising peaks at: 4.3° 2θ, 8.7° 2θ, and 20.6° 2θ, all ±0.2° 2θ. In one embodiment, the crystalline monosodium monohydrate salt has an XRPD spectrum in which the 10 most intense peaks include 5 or more peaks which have a 2θ value selected from: 4.3° 2θ, 6.2° 2θ, 6.7° 2θ, 7.3° 2θ, 8.7° 2θ, 9.0° 2θ, 12.1° 2θ, 15.8° 2θ, 16.5° 2θ, 18.0° 2θ, 18.1° 2θ, 20.6° 2θ, 21.6° 2θ, and 24.5° 2θ, all ±0.2° 2θ. The XRPD spectrum may be obtained as described in WO 2019/206871, which is incorporated in its entirety herein by reference.

In one embodiment, the crystalline monosodium monohydrate salt is as described in WO 2019/206871, which is incorporated in its entirety herein by reference. In one embodiment, the crystalline monosodium monohydrate salt has the polymorphic form described in WO 2019/206871, which is incorporated in its entirety herein by reference. In one embodiment, the crystalline monosodium monohydrate salt is prepared according to the method described in WO 2019/206871, which is incorporated in its entirety herein by reference.

Experimental—Prophylactic Efficacy

Objective

To determine the oral anti-inflammatory and anti-nociceptive efficacy of prophylactic treatment with the compound of formula (I) in a rodent model MSU-induced knee inflammation (gout) in male Wistar rats.

Protocol

A. Preparation of MSU crystals:
1. 4 g uric acid was dissolved in 800 ml 0.03 N NaOH and heated to 60° C. NaOH was added as required to maintain pH 8.9. The solution was stored overnight at 4-8° C.
2. Crystals were filtered, washed and dried.
3. Crystals were suspended in sterile saline just prior to use.

B. Efficacy Study:
1. The rats were distributed to 5 groups of 10 rats per group such that each group had a similar mean weight.
2. DAY −1. Baseline readings were taken

Baseline knee diameters were measured by digital caliper.

Baseline responses to mechanical allodynia, as measured by von Frey fibres, were recorded.
3. DAY 0:

a. The rats were weighed and dosed by oral gavage with vehicle, prednisolone (10 mg/kg) or the compound of formula (I) (1, 3 or 10 mg/kg)

b. One hour after dosing, rats in Groups 1-5 were anesthetized and injected IA into the left knee with 2 mg MSU in 50 μl saline.
4. DAY 1:

a. Rats were weighed and dosed as on DAY 0.

b. One hour after dosing, mechanical allodynia and knee diameters were measured and recorded.
5. DAY 2:

a. Rats were weighed and dosed as on DAY 0.

b. One hour after dosing, mechanical allodynia and knee diameters were measured and recorded.

c. Four hours after final dosing, terminal bleeds were collected.

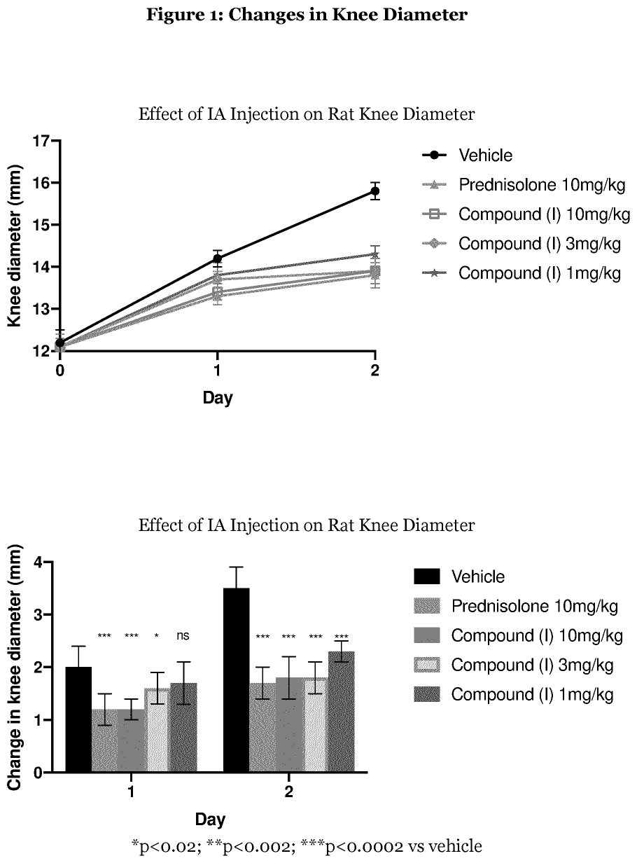

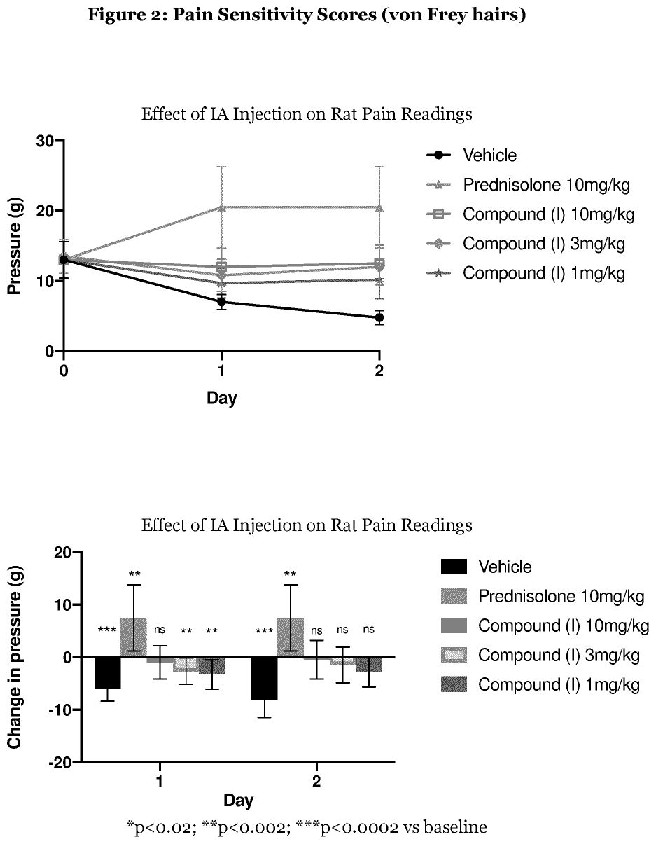

Results (FIGS. 1 and 2)

Disease Development

Once daily oral administration of 1 ml/kg of PBS had no effect on animal health. In the diseased rats an intra-articular injection of the left knee with 2 mg MSU resulted in a significant left knee diameter increase from 12.2 mm on DAY 0 to 15.8 mm on DAY 2. The response to mechanical allodynia in diseased rats fell from 13.0 g of pressure on DAY 0 to 4.8 g of pressure on DAY 2 indicating an increase in pain response.

Effect of Treatment with Prednisolone

Once daily oral administration of 10 mg/kg prednisolone, starting one hour prior to intra-articular challenge with 2 mg MSU had no significant effect on animal weight. This treatment resulted in a significant 52% inhibition of the MSU-stimulated knee edema. This treatment regimen also resulted in a significant insensitivity to pain as reflected by a significant increase in the amount of pressure needed to elicit a response.

Effect of Treatment with the Compound of Formula (I)

Once daily oral administration of the compound of formula (I) (sodium salt) in PBS, starting one hour prior to intra-articular challenge with 2 mg MSU had no significant effect on animal weight. This treatment regimen resulted in a significant dose-dependent inhibition of the MSU-stimulated response. At the highest dose (10 mg/kg, Group 3), the MSU-stimulated knee edema was reduced by 49%, similar to the effect observed with prednisolone. This dose of the compound of formula (I) also resulted in a complete blockade of the pain associated with the MSU-induced knee edema. There was no significant difference between the intermediate dose (3 mg/kg, Group 4) and the high dose of the compound of formula (I). At the lowest dose (1 mg/kg, Group 5) the knee edema was significantly reduced by 34% and the pain response was reduced by 66%. In a previous study, a 30 mg/kg oral dose of the compound of formula (I) gave similar efficacy to the 10 mg/kg dose.

### CONCLUSION

Daily oral administration with the compound of formula (I) resulted in a significant dose-dependent reduction in the MSU-induced edema and pain. At 3 mg/kg and 10 mg/kg, the effects were similar to that observed with 10 mg/kg prednisolone.

Experimental—Therapeutic Efficacy

Objective

To determine the oral anti-inflammatory and anti-nociceptive efficacy of therapeutic treatment with the compound of formula (I) in a rodent model MSU-induced knee inflammation (gout) in male Wistar rats.

Protocol

DAY 0

300 mg MSU crystal were suspended in 7.5 ml PBS (Sigma, Cat. D8662, lot. RNBG0405, exp. April 2019) to prepare a 40 mg/ml suspension. Material was kept in a sonicating water bath to maintain homogeneous suspension between injections. The rats were weighed.

Treatment Groups

| Group | Treatment | Dose | | Route |
|---|---|---|---|---|
| 1 | PBS | 1 | ml/kg | PO |
| 2 | Anakinra | 50 | mg/kg | IP |
| 3 | Compound of formula (I) | 10 | mg/kg | PO |

The rats were anesthetized (SOP 1810), knees were shaved, and injected into the left knee with 50 μl MSU (2 mg) crystals using a 25 G needle fitted to a 1 ml syringe.

DAY 1-3, Daily Dosing

Group 1=PBS (Sigma, Cat. D8662, lot RNBG0405, exp. April 2016). Dosed at 1 ml/kg, PO.

Group 2=Anakinra (100 mg/0.67 ml, Sobi, lot 31301-1B, exp. March 2020). Dosed at 0.335 ml/kg, IP.

Group 3=37 mg of the compound of formula (I) was dissolved in 3.7 ml PBS to prepare a 10 mg/ml solution. Dosed at 1 ml/kg, PO.

Test materials were made fresh daily. All rats were weighed and dosed by intraperitoneal injection or orally. Mechanical allodynia responses and knee diameters were recorded one hour post dosing.

On DAY 3, four hours after dosing, the rats were anesthetized and exsanguinated into pre-chilled K2 EDTA vacutainer tubes (Becton Dickinson, Cat. 367844, lot 6253682, exp. Jan. 31, 2018). The blood was processed to plasma, gently mixed and centrifuged at 4° C. 1800g for 10 minutes not later than 20 minutes from blood collection, which was stored in two 90 μl minimum volume and four 20 μl aliquots were prepared in labeled eppendorf tubes, and immediately stored frozen at −80° C. The carcasses were disposed of appropriately.

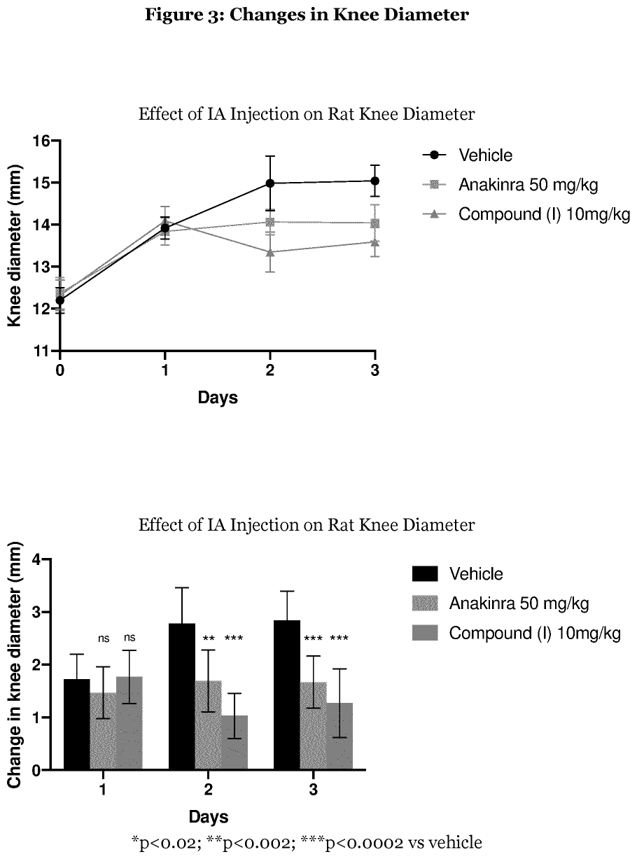

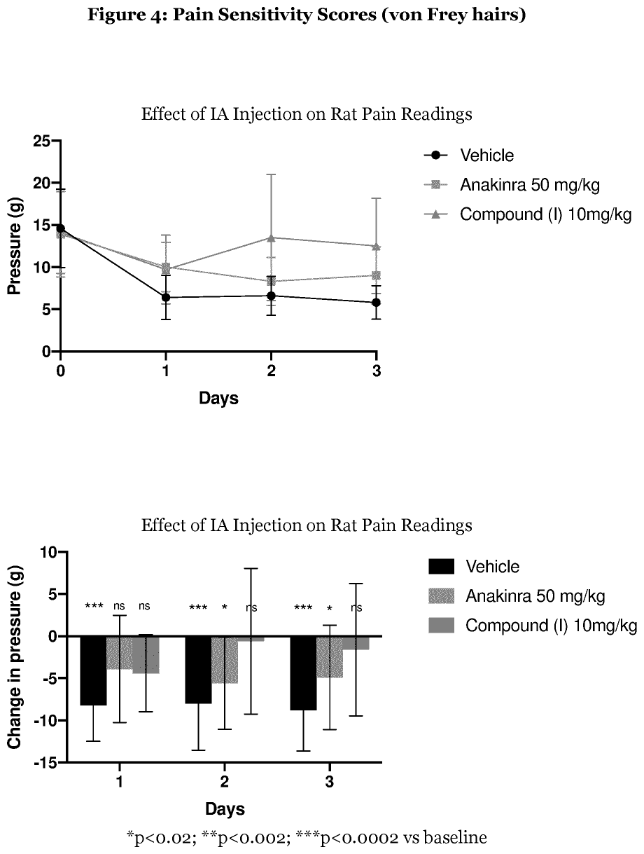

Results (FIGS. 3 and 4)

Disease Development:

Once daily oral administration of 1 ml/kg of PBS on Day 1, 2 and 3 had no effect on animal health. In the diseased rats an intra-articular injection of the left knee with 2 mg MSU resulted in a significant left knee diameter increase from 12.2 mm on the Baseline to 15.0 mm on DAY 3. The response to mechanical allodynia in diseased rats fell from 14.6 grams of pressure on the Baseline to 5.8 grams of pressure on DAY 3 indicating a higher sensitivity in pain response.

Effect of Treatment with Anakinra (Group 2):

Once daily intraperitoneal administration of 50 ml/kg of Anakinra on Day 1, 2 and 3 had no effect on animal health. In the Anakinra treated rats an intra-articular injection of the left knee with 2 mg MSU resulted in a left knee diameter increase from 12.4 mm on the Baseline to 14.0 mm on DAY 3. The response to mechanical allodynia in Anakinra treated rats increased from 13.9 grams of pressure on the Baseline to 9.0 grams of pressure on DAY 3 indicating a higher sensitivity in pain response. There is a significant statistical difference between Group 2 and Group 1 (disease—Vehicle).

Effect of Treatment with the Compound of Formula (I) (Group 3):

Once daily oral administration of 10 mg/kg of the compound of formula (I) on Day 1, 2 and 3 had no effect on animal health. In the rats treated with the compound of formula (I), an intra-articular injection of the left knee with 2 mg MSU resulted in a left knee diameter increase from 12.3 mm on the Baseline to 13.6 mm on DAY 3. The response to mechanical allodynia in the compound of formula (I) treated rats increased from 14.5 grams of pressure on the Baseline to 12.5 grams of pressure on DAY 3 indicating a higher sensitivity in pain response. There is a significant statistical difference between Group 3 and Group 1 (disease—Vehicle).

### CONCLUSION

Daily administration with the compound of formula (I) at 10 mg/kg orally and Anakinra at 50 mg/kg by peritoneal injection resulted in a significant reduction in the MSU-induced edema and pain.

1-16. (canceled)

17. A method for the treatment or prevention of arthritis in a patient in need thereof, wherein the method comprises administering to the patient in need thereof a therapeutically or prophylactically effective amount of a compound of formula (I): 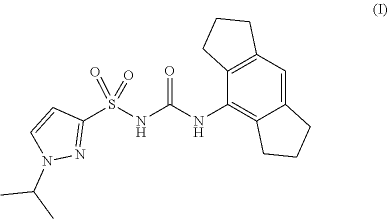 [SMILES: CC(C)n1ccc(S(=O)(=O)NC(=O)Nc2c3c(cc4c2CCC4)CCC3)n1]
or a pharmaceutically acceptable salt thereof.

18. The method as claimed in claim 17, wherein the arthritis is gout.

19. The method as claimed in claim 17, wherein the arthritis is pseudogout.

20. The method as claimed in claim 17, wherein the arthritis is rheumatoid arthritis.

21. The method as claimed in claim 17, wherein the arthritis is osteoarthritis.

22. The method as claimed in claim 17, wherein the treatment or prevention comprises the treatment or prevention of inflammation.

23. The method as claimed in claim 17, wherein the treatment or prevention comprises the oral administration of the compound or the salt thereof.

24. The method as claimed in claim 17, wherein the compound or salt is a sodium salt.

25. The method as claimed in claim 17, wherein the compound or salt is a monosodium salt.

26. The method as claimed in claim 17, wherein the compound or salt is a monohydrate.

27. The method as claimed in claim 17, wherein the compound or salt is crystalline.

28. The method as claimed in claim 17, wherein the compound or salt is a crystalline monosodium monohydrate salt.

29. The method as claimed in claim 28, wherein the crystalline monosodium monohydrate salt has an XRPD spectrum comprising peaks at: 4.3° 2θ, 8.7° 2θ, and 20.6° 2θ, all ±0.2° 2θ.

30. The method as claimed in claim 28, wherein the crystalline monosodium monohydrate salt has an XRPD spectrum in which the 10 most intense peaks include 5 or more peaks which have a 2θ value selected from: 4.3° 2θ, 6.2° 2θ, 6.7° 2θ, 7.3° 2θ, 8.7° 2θ, 9.0° 2θ, 12.1° 2θ, 15.8° 2θ, 16.5° 2θ, 18.0° 2θ, 18.1° 2θ, 20.6° 2θ, 21.6° 2θ, and 24.5° 2θ, all ±0.2° 2θ.

31. The method as claimed in claim 17, wherein the compound or the pharmaceutically acceptable salt thereof is administered as a pharmaceutical composition further comprising a pharmaceutically acceptable excipient.

32. The method as claimed in claim 31, wherein the pharmaceutical composition is suitable for oral administration.
